(19) United States
(12) Patent Application Publication
(10) Pub. No.: US 2023/0092867 A1
(43) Pub. Date: Mar. 23, 2023

(54) A NOVEL WASH BUFFER SOLUTION FOR AFFINITY CHROMATOGRAPHY

(71) Applicant: WUXI BIOLOGICS IRELAND LIMITED, Co Louth (IE)

(72) Inventors: Haikuan LIU, Shanghai (CN); Qian PANG, Shanghai (CN); Jing WU, Shanghai (CN)

(21) Appl. No.: 17/794,233

(22) PCT Filed: Jan. 19, 2021
(86) PCT No.: PCT/CN2021/072701

§ 371 Date: Jul. 20, 2022

(30) Foreign Application Priority Data
Jan. 20, 2020 (CN) PCT/CN2020/073298

(51) Int. Cl. C07K 1/22; C07K 14/765; B01D 15/38

(52) U.S. Cl. CPC C07K 1/22; C07K 14/765; B01D 15/3809; C07K 2319/30

(57) ABSTRACT

Provided is a method for improving impurities removal in the protein purification by affinity chromatography, comprising 1) loading a protein sample onto an affinity chromatography column, 2) washing the column with a wash buffer solution comprising Histidine or Imidazole, and a pH-adjusting agent.

### TECHNICAL FIELD

The present disclosure generally relates to a composition and a method of removing impurities when purifying protein sample.

### BACKGROUND

Protein A chromatography is generally regarded as a highly effective purification step due to the specific interaction between the protein A ligand and the monoclonal antibody. For this reason, it's routinely utilized for a direct capture step with subsequent polishing column to meet the purity requirements for a biopharmaceutical product. In this initial step, the high selectivity of Protein A resins leaves most of the non-target proteins in the flow-through. However, certain impurities including host cell protein (HCPs), high molecular weight ((HMWs) and low molecular weight (LMWs), may remain within the column along with the target protein. As the most effective unit operation for impurities removal in the downstream process, Protein A chromatography can remove >90% of the HCPs in the clarified media. Therefore, it's particularly important to optimize the removal of impurities during the affinity chromatography step.

Among impurities, HCPs are those produced or encoded by the organisms, and unrelated to the intended recombinant product. HCPs form a major class of process-related impurities and even at low levels they can potentially compromise the safety and efficacy of biopharmaceuticals. In addition to safety concerns, the presence of HCPs is also known to have an impact on product quality. As HCPs cause both safety and efficacy issues, it is desirable to have them removed as completely as possible via the downstream process.

Various wash solutions have been described for removal of impurities from Protein A columns, including wash solutions containing one of the following: hydrophobic electrolytes (e.g., tetramethylammonium chloride, tetraethylammonium chloride, tetrapropylammonium chloride or tetrabutylammonium chloride at pH 5.0-7.0), solvents (e.g., 5-20% isopropanol or polypropylene/hexylene glycol), urea (e.g., at a concentration of 1-4 M), detergents (e.g., 0.1-1% PS 20 or PS 80), polymers (e.g., 5-15% polyethylene glycol such as PEG400 or PEG8000) or highly concentrated buffer solutions such as Tris-HCI, acetate, sulfate, phosphate or citrate buffers at a concentration of 0.8-2.0 M and a pH between 5.0 and 7.0.

### SUMMARY OF THE DISCLOSURE

In one aspect, the disclosure provides a method for improving impurities removal in the protein purification by affinity chromatography, comprising the following steps:

1) loading a protein sample onto an affinity chromatography column,

2) washing the column with a wash buffer solution comprising a compound of the formula I and a pH-adjusting agent,

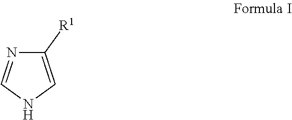

wherein, R¹ is H or C₁₋₆ alkyl; wherein C₁₋₆ alkyl is unsubstituted or substituted by one or two or three substituents independently selected from carboxy, amino, halogen or hydroxy.

In one embodiment, the compound is Histidine or Imidazole.

The disclosure also provides a method for improving impurities removal in the protein purification by affinity chromatography, comprising the following steps:

1) loading a protein sample onto an affinity chromatography column,

2) washing the column with a wash buffer solution comprises Serine and/or Cysteine, and a pH-adjusting agent.

In one embodiment, the affinity chromatography is selected from Protein A chromatography, Capto Blue (High Sub) chromatography, Protein G chromatography, Protein L chromatography, Lambda Fab Select chromatography, Kappa Select chromatography, 1 g Select chromatography, Blue Sepharose chromatography, Capto Heparin chromatography, VII Select chromatography, VIII Select chromatography, XSelect chromatography and Capto L chromatography.

In one embodiment, the pH-adjusting agent comprises acetate buffer such as NaAc and/or HAc, citrate buffer, Phosphate Buffer, or Tris-HCl.

In one specific embodiment, the percentage of molar mass of the compound in the volume of the wash buffer solution is about 100 mM and more, preferably is from about 100 mM to about 1 M, more preferably is from about 300 mM to about 700 mM, for examples, about 100 mM, about 200 mM, about 300 mM, about 400 mM, about 500 mM, about 600 mM, about 700 mM, about 800 mM, about 900 mM, or about 1 M.

In one specific embodiment, the pH of the wash buffer solution is about pH5.5 or less, for examples, about pH5.0, about pH4.5, about pH4.0, about pH3.5.

In another embodiment, the aforesaid method does not comprise elution step after the step 2).

In one specific embodiment, the protein sample is an antibody e.g. monoclonal antibody, or a fusion protein. The fusion protein is an Fe-fusion protein which contains an Fc domain recognizable by Protein A. The Fc-fusion protein is composed of an Fe domain of IgG linked to a peptide or protein of interest. In another specific embodiment, the fusion protein is a HAS (Human Serum Albumin)-fusion protein. The HAS-fusion protein is composed of a HAS linked to a peptide or protein of interest.

In one specific embodiment, the impurities comprise host cell proteins (HCPs).

In another aspect, the present disclosure provides a composition for use in improving impurities removal in the protein purification by affinity chromatography, wherein the composition comprises at least one compound of the Formula I:

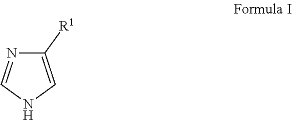

wherein, R¹ is H or C₁₋₆ alkyl; wherein C₁₋₆ alkyl is unsubstituted or substituted by one or two or three substituents independently selected from carboixy, amno, halogen or hydroxy.

In one embodiment, R¹ is H and the compound is Imidazole.

In one embodiment, R¹ is C₃ alkyl substituted by carboxy and amino.

In one embodiment, the compound is Histidine or Imidazole.

The present disclosure provides a composition for use in improving impurities removal in the protein purification by affinity chromatography, wherein the composition comprises Serine and/or Cysteine.

In one embodiment, the affinity chromatography is selected from Protein A chromatography, Capto Blue (High Sub) chromatography, Protein G chromatography, Protein L chromatography, Lambda Fab Select chromatography, Kappa Select chromatography, 1 g Select chromatography, Blue Sepharose chromatography, Capto Heparin chromatography, VII Select chromatography, VIII Select chromatography, XSelect chromatography and Capto L chromatography.

In one embodiment, the pH-adjusting agent comprises acetate buffer such as NaAc and/or HAc, citrate buffer, Phosphate Buffer, or Tris-HCl.

In another aspect, the disclosure provides a kit for use in improving impurities removal in the protein purification by affinity chromatography, wherein the kit comprises composition comprises a compound of the Formula I, Serine, or Cysteine:

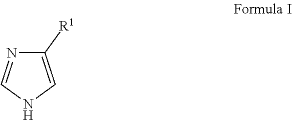

wherein, R¹ is H or C₁₋₆ alkyl; wherein C₁₋₆ alkyl is unsubstituted or substituted by one or two or three substituents independently selected from carboxy, amino, halogen or hydroxy.

In one embodiment, R¹ is H and the compound is Imidazole.

In one embodiment, R¹ is C₃ alkyl substituted by carboxy and amino.

In one embodiment, the compound comprises Histidine or Imidazole.

In one embodiment, the affinity chromatography is Protein A chromatography, Capto Blue (High Sub) chromatography, Protein G chromatography, Protein L chromatography, Lambda Fab Select chromatography, Kappa Select chromatography, 1 g Select chromatography, Blue Sepharose chromatography, Capto Heparin chromatography, VII Select chromatography, VIII Select chromatography, XSelect chromatography and Capto L chromatography.

In one embodiment, the kit further comprises a pH-adjusting agent.

In one embodiment, the pH-adjusting agent comprises acetate buffer such as NaAc and/or HAc, citrate buffer, Phosphate Buffer, or Tris-HCl.

The disclosure provides the use of the aforementioned composition in preparation of wash solution for improving impurities removal in the protein purification by affinity chromatography.

### THE FEATURES AND ADVANTAGES

This disclosure provides an efficient and robust wash solution for affinity chromatography. The wash solution is characterized by the presence of Histidine or Imidazole (an aromatic heterocycle, functional group of Histidine), applying in a washing step prior to the elution step without compromising the product recovery.

### DETAILED DESCRIPTION

In order that the present disclosure may be more readily understood, certain terms are first defined. Additional definitions are set forth throughout the detailed description. It is to be understood that the terminology used herein is for the purpose of describing particular embodiments only, and is not intended to be limiting.

As used in this specification and the appended claims, the singular forms “a”, “an”, and “the” include plural referents unless the content clearly dictates otherwise. Thus, for example, reference to “a polypeptide” includes a combination of two or more polypeptides, and the like.

“About” as used herein when referring to a measurable value such as an amount, a temporal duration, and the like, is meant to encompass variations of ±20% or ±10%, including ±5%, ±1%, and ±0.1% from the specified value, as such variations am appropriate to perform the disclosed methods.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, the preferred materials and methods are described herein. In describing and claiming the present disclosure, the following terminology will be used.

The term “protein sample” employed in the present disclosure refers to a protein which contains an Fe domain recognizable by Protein A. Such protein comprises antibodies and Fc-fusion proteins. The antibody could be a monoclonal antibody, or a polyclonal antibody. The antibody could be monospecific, bispecific or multi-specific. The antibody could be a mouse antibody, a chimeric antibody, a humanized antibody or a human antibody. The antibody could be a natural antibody or a recombinant antibody. An Fe-fusion protein is composed of an Fc domain of an antibody and a genetically linked active protein.

“Polypeptide,” “peptide” and “protein” are used interchangeably herein to refer to a polymer of amino acid residues. A polypeptide can be of natural (tissue-derived) origins, recombinant or natural expression from prokaryotic or eukaryotic cellular preparations, or produced chemically via synthetic methods. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid mimetics refers to chemical compounds that have a structure that is different from the general chemical structure of an amino acid, but that functions in a manner similar to a naturally occurring amino acid. Non-natural residues arm well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-4-isopropyl)-phenylglycine: D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine: D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

“Peptide” as used herein includes peptides which are conservative variations of those peptides specifically exemplified herein. “Conservative variation” as used herein denotes the replacement of an amino acid residue by another, biologically similar residue. Examples of conservative variations include, but are not limited to, the substitution of one hydrophobic residue such as Isoleucine, Valine, Leucine, Alanine, Cysteine, Glycine, Phenylalanine, Proline, Tryptophan, Tyrosine, Norleucine or Methionine for another, or the substitution of one polar residue for another, such as the substitution of Arginine for Lysine, Glutamic for Aspartic acids, or Glutamine for Asparagine, and the like. Neutral hydrophilic amino acids which can be substituted for one another include Asparagine, Glutamine, Serine and Threonine. “Conservative variation” also includes the use of a substituted amino acid in place of an unsubstituted parent amino acid provided that antibodies raised to the substituted polypeptide also immunoreact with the unsubstituted polypeptide. Such conservative substitutions are within the definition of the classes of the peptides of the disclosure. “Cationic” as used herein refers to any peptide that possesses a net positive charge at pH 7.4. The biological activity of the peptides can be determined by standard methods known to those of skill in the art and described herein.

The term “Fc domain” employed in the present disclosure refers to the fragment crystallizable region of an antibody. Fe domain is derived from the constant domains of the antibody's heavy chains. The “Fc domain” can be recognized and bound by Protein A.

In one embodiment, the protein is an antigen binding protein. In one embodiment, the antigen binding protein is an antibody. In one embodiment the antibody is of the IgG class. In one embodiment, the antigen binding protein is an immunoglobulin single variable domain.

Exemplary antibodies that could be used in the present disclosure include Adalimumab, Bezlotoxumab, Avelumab, Dupilumab, Durvalumab, Ocrelizumab, Brodalumab, Reslizumab, Olaratumab, Daratumnumab, Elotuzunab, Necitumumab, Infliximab, Obiltoxaximab, Atezolizumab, Secukinumab, Mepolizumab, Nivolumab, Alirocumab, Evolocumab, Dinutuximab, Bevacizumab, Pembrolizumab, Ramucirumab, Vedolizumab, Siltuximab, Alemtuzumab, Trastuzumab, Pertuzumab, Infliximab, Obinutuzumab, Brentuximab, Raxibacumab, Belimumab, Ipilimumab, Denosumab, Ofatumumab, Besilesomab, Tocilizumab, Canakinumab, Golimumab, Ustekinumab, Certolizumab, Catumaxomab, Eculizumab, Ranibizumab, Panitumumab, Natalizumab, Catumaxomab, Bevacizumab, Omalizumab, Cetuximab, Efalizumab, Ibritumomab, Fanolesomab, Tositumomab, Alemtuzumab, Trastuzumab, Gemtuzumab, Infliximab, Palivizumab, Necitumumab, Basiliximab, Rituximab, Capromab, Satumomab, Muromonab, etc.

Exemplary Fc-fusion proteins that could be used in the present disclosure include Etanercept, Alefacept, Abatacept, Rilonacept, Romiplostim, Belatacept, Aflibercept, etc.

The term“chromatography” refers to any kind of technique which separates an analyte of interest (e.g., an Fe domain containing protein such as an immunoglobulin) from other molecules present in a mixture. Usually, the analyte of interest is separated from other molecules as a result of differences in rates at which the individual molecules of the mixture migrate through a stationary medium under the influence of a moving phase, or in bind and elute processes.

The term “Protein A” employed in the present disclosure encompasses Protein A recovered from a native source, Protein A produced synthetically (e.g., by peptide synthesis or by recombinant techniques), and functional variants thereof. Protein A exhibits high affinity for an Fe domain. Protein A can be purchased commercially from Repligen, Pharmacia and Fermatech. Protein A is generally immobilized on a solid phase support material. The term “Protein A” also refers to an affinity chromatography resin or column containing chromatographic solid support matrix to which Protein A is covalently attached.

A “buffer” is a solution that resists changes in pH by the action of its acid-base conjugate components. Various buffers which can be employed depending, for example, on the desired pH of the buffer are described in “Buffers. A Guide for the Preparation and Use of Buffers in Biological Systems, Gueffroy, D., ed. Calbiochem Corporation, 1975”. In some steps of the methods of the claimed disclosure, a buffer has a pH in the range from 2.0 to 4.0, or from 2.8 to 3.8. In other steps of the claimed disclosure, a buffer has a pH in the range of 5.0 to 9.0. In other steps of the claimed disclosure, a buffer has a pH in the range of 4.0 to 6.5. In yet other steps of the methods of the claimed disclosure, a buffer has a pH lower than 4.0. Non-limiting examples of buffers that will control the pH in this range include MES, MOPS, MOPSO, Tris, HEPES, phosphate, acetate, citrate, succinate, and ammonium buffers, as well as compositions of these. The term “pH-adjusting agent” is a buffer solution that is capable of producing a selected pH of between about 1.0 and about 14.0 in the aqueous solution. pH-adjusting agent may be acetate buffer such as NaAc and/or HAc, citrate buffer, Phosphate Buffer, or Tris-HCl.

The term “wash buffer” refers to the buffer used to wash the chromatography column post sample loading and prior to elution.

The term “elution buffer” refers the buffer used to elute the target protein from the solid phase. The conductivity and/or pH of the elution buffer is/are usually such that the target protein is eluted from the chromatography resin.

### Materials

Sodium acetate trihydrate, sodium chloride, sodium hydroxide, Tris (hydroxymethyl) aminomethane, Sodium dihydrogen phosphate and disodium phosphate were purchased from Merck (Darmstadt, Germany). Acetic Acid, L-Histidine, L Histidine Monohydrochloride, L-Arginine Hydrochloride, L-Cysteine, Serine, Proline and Hydrochloric Acid (6.0N Solution) were purchased from J. T. Baker, Millipore (Bedford, Mass., America). Imidazole were purchased from Sigma (Saint Louis, America).

### Equipment

An AKTA pure 150 system installed with Unicorn software version 6.3 (GE Healthcare, Uppsala, Sweden) was used for all chromatographic nus. pH and conductivity was measured using SevenExcellence S470 pH/Conductivity meter (Mettler-Toledo, Columbus, Ohio, USA). Protein concentration was measured using a NanoDrop One spectrophotometer (Thermo Fisher Scientific, Waltham, Mass., USA). An Agilent 1260 liquid chromatography instrument (Agilent Technologies, Santa Clara, Calif., USA) was used for SEC-HPLC analysis.

### Methods

### Protein A/Capto Blue Chromatography

Eshmuno A (Protein A affinity medium)/Capto Blue (high-sub) was packed in a 0.5 cm diameter column with 15 cm bed height. The column volume (CV) is approximately 3 ml. The load is the culture harvest clarified. For all runs, the column was loaded and run in bind-elute mode. The target protein was eluted with elution buffer. For all runs, after sample loading the column was washed with different buffer for 3 CV prior to elution. For all chromatographic runs, the system was run at a flow rate of 180 cm/hr (residence time: 5 min). All chromatograms were recorded by monitoring UV absorbance at 280 nm Elution from selected runs was collected in fractions and analyzed by SEC-HPLC for monomer purity, HCP and/or PLBL2 assay.

### Size-Exclusion Chromatography-High Performance Liquid Chromatography (SEC-HPLC)

All samples were analysed using a Tosoh TSKgel G3000SWx1 stainless steel column (7.8×300 mm). 100 μg of sample was injected per run. The mobile phase consisted of 50 mM sodium phosphate, 300 mM sodium chloride at pH 6.8. Each sample was eluted isocratically for 20 min at a flow rate of 1.0 mL/min. Protein elution was monitored by UV absorbance at 280 nm. The peaks corresponding to the monomer and aggregates were integrated to calculate the percentage of each species.

### Host Cell Protein (HCP) Assay

Determination of residual CHO host cell protein is achieved by using CHO Host Cell Proteins F550 kit from Cygnus Technologies. Samples containing CHO HCPs react simultaneously with a HRP labelled anti-CHO antibody and capture anti-CHO antibody coated in microtiter strips. The immunological reactions result in the formation of a sandwich complex of solid phase antibody-HCP-enzyme labelled antibody. The substrate, TMB then react with HRP. Terminate the reaction and read the OD value in 450 nm and 650 nm. The OD values determined are directly proportional to the concentration of CHO HCPs present in samples to determine the level of residual CHO host cell protein present in samples.

### Hamster Phospholipase B-Like 2(PLBL2) Assay

Samples are analyzed using Hamster phospholipase B-like (PLBL2) ELISA kit from MyBioSource. In this assay the PLBL2 present in samples reacts with the anti-PLBL2 antibodies which have been adsorbed to the surface of polystyrene microtiter wells. After the removal of unbound proteins by washing, the Detection Antibody, biotin conjugated anti-PLBL2 is added and complexes are formed. Following a wash step, the horseradish peroxidase (HRP) conjugated Streptavidin is added and complexes are formed. Following another washing step, the complexes are assayed by the addition of TMB. The quantity of PLBL2 in the test sample can be interpolated from the standard curve constructed from the standards, and corrected for sample dilution.

### A Method

The disclosure provides a method for improving impurities removal in the protein purification by affinity chromatography, comprising the following steps:

1) loading a protein sample onto an affinity chromatography column,

2) washing the column with a wash buffer solution comprises at least one compound of the formula I and a pH-adjusting agent,

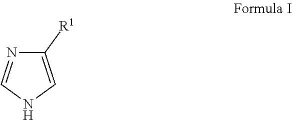

wherein R¹ is H or C₁₋₆ alkyl; wherein C₁₋₆ alkyl is unsubstituted or substituted by one or two or three substituents independently selected from carboxy, amino, halogen or hydroxy.

In one embodiment, the composition comprises Histidine and/or Imidazole.

The disclosure also discloses a method for improving impurities removal in the protein purification by affinity chromatography, comprising the following steps:

1) loading a protein sample onto an affinity chromatography column,

2) washing the column with a wash buffer solution comprises Sine and/or Cysteine, and a pH-adjusting agent.

In one embodiment, the affinity chromatography is selected from Protein A chromatography, Capto Blue (High Sub) chromatography, Protein G chromatography, Protein L chromatography, Lambda Fab Select chromatography, Kappa Select chromatography, Ig Select chromatography, Blue Sepharose chromatography, Capto Heparin chromatography, VII Select chromatography, VIIH Select chromatography, XSelect chromatography and Capto L chromatography.

In one embodiment, the pH-adjusting agent comprises acetate buffer such as NaAc and/or HAc, citrate buffer, Phosphate Buffer, or Tris-HCl.

In one specific embodiment, the percentage of molar mass of the compound in the volume of the wash buffer solution is about 100 mM and more, preferably is from about 100 mM to about 1 M, more preferably is from about 100 mM to about 900 mM, 100 mM to about 800 mM, 100 mM to about 700 mM, 100 mM to about 600 mM, 100 mM to about 500 mM, 100 mM to about 400 mM, 100 mM to about 300 mM, 100 mM to about 200 mM, 200 mM to about 1 M, 300 mM to about 1 M, 400 mM to about 1 M, 500 mM to about 1M, 600 mM to about 1 M, 700 mM to about 1 M, 800 mM to about 1 M, 900 mM to about t M, 250 mM to about 750 mM, or 500 mM.

In one specific embodiment, the pH of the wash buffer solution is about pH5.5 or less, pH5 or less, pH4.5 or less, pH4 or less, pH3.5 or less, pH3 or less. In another embodiment, the aforesaid method does not comprise elution step after the step 2).

In one specific embodiment, the protein sample is an antibody e.g. monoclonal antibody, or a fusion protein. The fusion protein is an Fc-fusion protein which contains an Fc domain recognizable by Protein A. The Fc-fusion protein is composed of an Fe domain of IgG linked to a peptide or protein of interest. In one another specific embodiment the fusion protein is a HAS (Human Serum Albumin)-fusion protein. The HAS-fusion protein is composed of a HAS linked to a peptide or protein of interest.

In one specific embodiment, the impurities comprise host cell proteins (HCPs).

### Composition

In one aspect, the present disclosure provides a composition for improving impurities removal in the protein purification by affinity chromatography, wherein the composition comprises at least one compound of the Formula I:

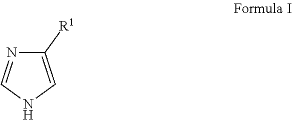

wherein, R¹ is H or C₁₋₆ alkyl; wherein C₁₋₆ alkyl is unsubstituted or substituted by one or two or three substituents independently selected from carboxy, amino, halogen or hydroxy. In one embodiment, R¹ is H and the compound is Imidazole. In one embodiment, R¹ is C₃ alkyl substituted by carboxy and amino. In one embodiment, the composition comprises Histidine and/or Imidazole.

The present disclosure provides a composition for improving impurities removal in the protein purification by affinity chromatography, wherein the composition comprises Serine and/or Cysteine.

In one embodiment, the affinity chromatography is selected from Protein A chromatography, Capto Blue (High Sub) chromatography, Protein G chromatography, Protein L chromatography, Lambda Fab Select chromatography, Kappa Select chromatography, 1 g Select chromatography, Blue Sepharose chromatography, Capto Heparin chromatography, VII Select chromatography, VIII Select chromatography, XSelect chromatography and Capto L chromatography.

The disclosure provides the use of the composition in preparation of wash solution for improving impurities removal in the protein purification by affinity chromatography.

### A Kit

In another aspect, the disclosure provides a kit for improving impurities removal in the protein purification by affinity chromatography, wherein the kit comprises composition comprises a compound of the Formula I:

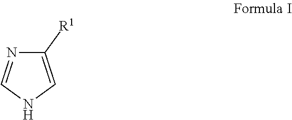

wherein R¹ is H or C₁₋₆ alkyl; wherein C₁₋₆ alkyl is unsubstituted or substituted by one or two or three substituents independently selected from carboxy, amino, halogen or hydroxy. In one embodiment, R¹ is H and the compound is Imidazole. In one embodiment, R¹ is C₃ alkyl substituted by carboxy and amino. In one embodiment, the composition comprises Histidine and/or Imidazole. In one embodiment, the affinity chromatography is selected from Protein A chromatography, Capto Blue (High Sub) chromatography, Protein G chromatography, Protein L chromatography, Lambda Fab Select chromatography, Kappa Select chromatography, 1 g Select chromatography, Blue Sepharose chromatography, Capto Heparin chromatography, VII Select chromatography, VIII Select chromatography, XSelect chromatography and Capto L chromatography. In one embodiment, the kit further comprises a pH-adjusting agent. In one embodiment, the pH-adjusting agent comprises acetate buffer such as NaAc and/or HAc, citrate buffer, Phosphate Buffer, or Tris-HCl.

The present disclosure provides a kit for improving impurities removal in the protein purification by affinity chromatography, wherein the kit comprises Serine and/or Cysteine.

### EXAMPLES

### Example 1: Comparison of Histidine Wash Solution to Other Wash Solutions

The cDNA sequence to express an anti-hTNFα as disclosed in U.S. Pat. No. 6,090,382 was cloned into two vectors, which contained Blasticidin and Zeocin resistance markers, respectively. Stable transfection was performed using liposome. After transfection, cells were passaged in selective media (CD CHO media containing 9 μg/mL Blasticidin and 400 μg/mL Zeocin) for pool selection. After about 2 weeks of pool selection, the pools were cloned by FACS sorting. The clones were screened by fed-batch cultures in spin tubes. The selected cell clones are cultured, and harvest is clarified from anti-hTNFα IgG4 containing cell culture.

In this example, the effect of Histidine containing wash solution on the removal of impurities from clarified harvest from IgG4 (anti-hTNFα) containing cell culture during affinity chromatography is assessed. Specifically, three wash solutions are compared: one containing 1 M NaCl at pH 5.5, the second containing 0.5 M Histidine at pH 5.5, the third containing 0.5 M Arginine at pH 5.5.

Clarified harvest from cell culture supernatants containing IgG4 is harvested by centrifugation and purified using an AC column, in particular a Protein A column (Millipore, Eshmuno A, 1 CV: 3 mL), according to the conditions described below in Table 1. The load capacity is 30 g/L.

TABLE 1
Operating Conditions for Protein A Column

| Step | Buffer | Rt (min) | (CV) Vol. |
|---|---|---|---|
| Rinse | 50 mM Tris-HAc, | | 2 |
| | 150 mM NaCl, pH 7.4 | | |
| Sanitization | 0.1M NaOH | | 3 |
| Equilibration | 50 mM Tris-HAc, | | 3 |
| | 150 mM NaCl pH 7.4 | | |
| Loading | NA | | NA |
| Wash 1 | 50 mM Tris-HAc, | 5 | 3 |
| | 150 mM NCl, pH 7.4 | | |
| Wash 2 | Variable | | 3 |
| Wash 3 | 50 mM NaAc-HAc, pH 5.5 | | 3 |
| Elution | 50 mM NaAc-HAc, pH 3.5 | | 50-50 mAu/mm |
| Strip | 1M Acetic Acid | | 3 |

The equilibrated column is loaded with clarified harvest and is first washed with wash 1 solution, followed by a second wash with wash 2 solution described in Table 2, and then eluted at low pH. The eluate is analyzed for its antibody concentration by UV 280, for HMW/LMW by analytical size exclusion chromatography (SEC) and for HCP content by enzyme-linked immunosorbent assay. The various wash solutions compared for the second wash are shown in Table 2.

TABLE 2
Wash Solutions with Varying Components for Second Wash

| Buffer | CV |
|---|---|
| Control (without wash 2) | 3 |
| 50 mM NaAc-HAc, 1M NaCl pH 5.5 | 3 |
| 50 mM NaAc-HAc, 500 mM Hisitidine-HCl, pH 5.5 | 3 |
| 50 mM NaAc-HAc, 500 mM Arginine-HCl, pH 5.5 | 3 |

The process performance for the Protein A purification of monoclonal antibody using the three different wash solutions are shown in Table 3.

TABLE 3
Comparison of Histidine Wash Solution to Other Wash Solutions

| | Yield | HMW | Main peak | LMW | HCP | PLBL2 |
|---|---|---|---|---|---|---|
| Buffer | (%) | (%) | (%) | (%) | (ppm) | (ppm) |
| Control (without wash 2) | 105 | 14.5 | 85.5 | 0.1 | 34409 | 8 |
| 50 mM NaAc—HAc, 1M NaCl, pH5.5 | 105 | 14.6 | 85.5 | 0.0 | 27517 | 5 |
| 50 mM NaAc—HAc, 500 mM Histidine-HCl, pH5.5 | 104 | 14.4 | 85.5 | 0.1 | 14522 | <2 |
| 50 mM NaAc—HAc, 500 mM Arginine-HCl, pH5.5 | 102 | 14.1 | 85.7 | 0.0 | 13340 | <2 |

As shown in Table 3, the results demonstrate that different wash 2 solutions have no effect on step yield and aggregate level while maintaining the higher recovery of product. Also, it should be noticed that NaCl wash showed no significant decrease in HCPs compared with control. However, the Histidine and Arginine wash solutions showed significant HCPs and PLBL2 removal compared to the NaCl wash and Control. Moreover, Histidine showed the similar ability in HCPs removal with Arginine solution, while Histidine wash has not been reported yet. Specifically, Histidine wash results in acceptable recovery of >95%, while providing a 2.5-fold reduction of HCPs compared with Control.

### Example 2: Comparison of Varying Histidine Concentration in Wash Solution

In this example, the concentration effect of Histidine-containing wash solution on the removal of impurities from an IgG4-containing cell culture during affinity chromatography is investigated. Four different concentration of Histidine in wash 2 solutions are compared.

Clarified cell culture supernatants containing IgG4 is harvested by centrifugation and purified using an AC column, in particular a Protein A column (Millipore, Eshmuno A, 1 CV: 3 mL), according to the conditions described in Table 2.

The equilibrated column is loaded with clarified harvest and is first washed with wash 1 solution, followed by a second wash with wash 2 solution described in Table 5 below, and then eluted at low pH. The eluate is analyzed for its antibody concentration by UV280, for HMW/LMW by analytical size exclusion chromatography (SEC) and for HCP content by enzyme-linked immunosorbent assay. The various wash solutions compared for the second wash are set forth below in Table 4.

TABLE 4
Wash Solution with Varying Histidine Concentration for Wash 2

| Buffer | CV |
|---|---|
| Control (without wash 2) | 3 |
| 50 mM NaAc-HAc, 100 mM Histidine-HCl, pH 5.5 | 3 |
| 50 mM NaAc-HAc, 300 mM Histidine-HCl, pH 5.5 | 3 |
| 50 mM NaAc-HAc, 500 mM Histidine-HCl, pH 5.5 | 3 |
| 50 mM NaAc-HAc, 700 mM Histidine-HCl, pH 5.5 | 3 |

The process performance for the Protein A purification of monoclonal antibody using the four different concentration of Histidine-containing wash solutions are shown in Table 5.

TABLE 5
Comparison of Varying Histidine Concentration for Wash 2

| | Yield | HMW | Main peak | LMW | HCP | PLBL2 |
|---|---|---|---|---|---|---|
| Buffer | (%) | (%) | (%) | (%) | (ppm) | (ppm) |
| Without wash 2 | 105 | 14.5 | 85.5 | 0.1 | 34409 | 8 |
| 50 mM NaAc—HAc, 100 mM Histidine-HCl, pH5.5 | 108 | 14.1 | 85.9 | 0.1 | 25537 | 4 |
| 50 mM NaAc—HAc, 300 mM Histidine-HCl, pH5.5 | 106 | 14.2 | 85.9 | 0.0 | 18154 | 2 |
| 50 mM NaAc—HAc, 500 mM Histidine-HCl, pH5.5 | 104 | 14.4 | 85.5 | 0.1 | 14522 | <2 |
| 50 mM NaAc—HAc, 700 mM Histidine-HCl, pH5.5 | 103 | 14.2 | 85.7 | 0.1 | 13463 | <2 |

As shown in Table 5, the results demonstrate that washing solution containing Histidine has no effect on yield compared with the non-Histidine containing buffer. Also, it is evident that the Histidine solution is efficient in removing HCPs and PLBL2. Higher Histidine concentration of wash solution results in lower HCP and PLBL2 in the eluate pool. Specifically, using a Histidine-containing wash solution at high concentration of 700 mM results in 3-fold reduction of HCPs, at least 4-fold reduction of PLBL2, as compared to a non-Histidine containing buffer at pH5.5.

### Example 3: Comparison of Varying Imidazole Concentration in Wash Solution

In this example, the concentration effect of Imidazole-containing wash buffer on the removal of impurities from an IgG4-containing cell culture during affinity chromatography is investigated. Specifically, four varying concentration of wash solutions are compared.

Clarified cell culture supernatants containing IgG4 is harvested by centrifugation and purified using an AC column, in particular a Protein A column (Millipore, Eshmuno A, 1 CV: 3 mL), according to the conditions described in Table 6.

TABLE 6
Operating Conditions for Protein A Column

| Step | Buffer | Rt (min) | (CV) Vol. |
|---|---|---|---|
| Rinse | 50 mM Tris-HAc, | | 2 |
| | 150 mM NaCl, pH 7.4 | | |
| Sanitization | 0.1M NaOH | | 3 |
| Equilibration | 50 mM Tris-HAc, | | 3 |
| | 150 mM NaCl, pH 7.4 | | |
| Loading | NA | | NA |
| Wash 1 | 50 mM Tris-HAc, | 5 | 3 |
| | 150 mM NaCl, pH 7.4 | | |
| Wash 2 | Variable (see Table 7) | | 3 |
| Wash 3 | 50 mM NaAc-HAc, pH 5.5 | | 3 |
| Elution | 50 mM NaAc-HAc, pH 3.5 | | 50-50 mAu/mm |
| Strip | 1M Acetic Acid | | 3 |

The equilibrated column is loaded with clarified harvest and is first washed with wash 1 solution, followed by a second wash with wash 2 solutions described in Table 7 below, and then eluted at low pH. The eluate is analyzed for its antibody concentration by UV280, for HMW/LMW by analytical size exclusion chromatography (SEC) and for HCP content by enzyme-linked immunosorbent assay, developed on the same cell line. The various wash solutions compared for the second wash are set forth in Table 8.

TABLE 7
Wash Solution with Varying Imidazole Concentration for Wash 2

| Buffer | CV |
|---|---|
| Without wash 2 | 3 |
| 50 mM NaAc-HAc, 100 mM Imidazole-HCl, pH 5.5 | 3 |
| 50 mM NaAc-HAc, 300 mM Imidazole-HCl, pH 5.5 | 3 |
| 50 mM NaAc-HAc, 500 mM Imidazole-HCl, pH 5.5 | 3 |
| 50 mM NaAc-HAc, 700 mM Imidazole-HCl, pH 5.5 | 3 |

The process performance for the Protein A purification of monoclonal antibody using the four different concentration of Histidine-containing wash solutions shown in Table 8.

TABLE 8
Comparison of Varying Imidazole Concentration in Wash Solution

| | Yield | HMW | Main peak | LMW | HCP | PLBL2 |
|---|---|---|---|---|---|---|
| Buffer | (%) | (%) | (%) | (%) | (ppm) | (ppm) |
| Without wash 2 | 105 | 14.5 | 85.5 | 0.1 | 34409 | 8 |
| 50 mM NaAc—HAc, 100 mM Imidazole-HCl, pH5.5 | 109 | 14.0 | 86.0 | 0.0 | 29245 | 4 |
| 50 mM NaAc—HAc, 300 mM Imidazole-HCl, pH5.5 | 104 | 15.1 | 84.8 | 0.1 | 14853 | <2 |
| 50 mM NaAc—HAc, 500 mM Imidazole-HCl, pH5.5 | 103 | 14.3 | 85.7 | 0.0 | 12070 | <2 |
| 50 mM NaAc—HAc, 700 mM Imidazole-HCl, pH5.5 | 105 | 15.4 | 84.4 | 0.0 | 11473 | <2 |

As shown in Table 8, the results demonstrate that washing solution containing Imidazole has no effect on step yield compared with the non-Imidazole containing buffer. Also, it is evident that the Imidazole-containing solution is efficient in removing HCPs and PLBL2. Higher Imidazole concentration of wash solution results in lower HCP in the eluate pool. Specifically, using an Imidazole-containing wash solution at high concentration of 700 mM results in 3-fold reduction of HCPs, at least 4-fold reduction of PLBL2, as compared to the non-Imidazole containing buffer at pH5.5. Compared with the data showed in last table, it's apparent that Imidazole (an aromatic heterocycle, functional group of Histidine) are more efficient in removing HCPs than Histidine.

### Example 4: Comparison of Acidic to Basic and Physiological pH in Histidine-Containing Wash Solution

In this example, the pH effect of Imidazole-containing wash buffer on the removal of impurities from an IgG4-containing cell culture during affinity chromatography is investigated. Specifically, four varying pH of wash solutions are compared.

Clarified cell culture supernatants containing IgG4 is harvested by centrifugation and purified using an AC column, in particular a Protein A column (Millipore, Eshmuno A, 1 CV: 3 mL), according to the conditions described below in Table 9.

TABLE 9
Operating Conditions for Protein A Column

| Step | Buffer | Rt (min) | (CV) Vol. |
|---|---|---|---|
| Rinse | 50 mM Tris-HAc, | | 2 |
| | 150 mM NaCl, pH 7.4 | | |
| Sanitization | 0.1M NaOH | | 3 |
| Equilibration | 50 mM Tris-HAc, | | 3 |
| | 150 mM NaCl, pH 7.4 | | |
| Loading | NA | | NA |
| Wash 1 | 50 mM Tris-HAc, | 5 | 3 |
| | 150 mM NaCl, pH 7.4 | | |
| Wash 2 | Variable (see Table 10) | | 3 |
| Wash 3 | 50 mM NaAc-HAc, pH 5.5 | | 3 |
| Elution | 50 mM NaAc-HAc, pH 3.5 | | 50-50 mAu/mm |
| Strip | 1M Acetic Acid | | 3 |

The equilibrated column is loaded with clarified harvest and is first washed with wash 1 solution, followed by a second wash with wash 2 solutions described in Table 10 below, and then eluted at low pH, The eluate is analyzed for its antibody concentration by UV280, for HMW/LMW by analytical size exclusion chromatography (SEC) and for HCP content by enzyme-linked immunosorbent assay. The various wash solutions compared for the second wash are set forth below in Table 10.

TABLE 10
Histidine Containing Wash Solutuon with Varying pH Values

| Buffer | CV |
|---|---|
| 50 mM NaAc-HAc, pH 5.0 | 3 |
| 50 mM NaAc-HAc, 250 mM Histidine-HCl, pH 5.0 | 3 |
| 50 mM PB, pH 7.0 | 3 |
| 50 mM PB, 250 mM Histidine-HCl, pH 7.0 | 3 |
| 50 mM Tris-HCl, pH 9.0 | 3 |
| 50 mM Tris-HCl, 250 mM Histidine-HCl, pH 9.0 | 3 |

The process performance for the Protein A purification of monoclonal antibody using the three different pH of Histidine-containing wash solutions shown in Table 11.

TABLE 11
Comparison of Acidic to Basic and Physiological pH in Histidine-containing Wash Solution

| | Yield | HMW | Main peak | LMW | HCP | PLBL2 |
|---|---|---|---|---|---|---|
| Buffer | (%) | (%) | (%) | (%) | (ppm) | (ppm) |
| 50 mM NaAc—HAc, pH5.0 | 103 | 14.1 | 85.9 | 0.0 | 35766 | 5 |
| 50 mM NaAc—HAc, 250 mM Histidine-HCl, pH5.0 | 107 | 13.6 | 86.2 | 0.0 | 23728 | 3 |
| 50 mM PB, pH7.0 | 109 | 13.9 | 86.0 | 0.0 | 29306 | 4 |
| 50 mM PB, 250 mM Histidine-HCl, pH7.0 | 107 | 14.7 | 85.2 | 0.1 | 31517 | 3 |
| 50 mM Tris-HCl, pH9.0 | 112 | 14.5 | 85.4 | 0.0 | 17800 | 3 |
| 50 mM Tris-HCl, 250 mM Histidine-HCl, pH9.0 | 108 | 14.7 | 85.2 | 0.1 | 18641 | <2 |

As shown in Table 11, varying the pH from acidic (pH 5.0) to basic (pH 9.0) has no effect on yield and aggregate level compared with the non-Histidine containing wash solution. It's evident that the Histidine-based solution is significantly efficient in removing HCPs at low pH, emphasizing the efficacy of the Histidine-containing wash solution at acidic pH for affinity chromatography. Furthermore, in this case, it is clear that Histidine alone does result in the desired HCP removal and it's only effective in composition with low pH.

### Example 5: Comparison of Acidic to Bask and Physiological pH in Imidazole-Containing Wash Buffer

In this example, the pH effect of imidazolo-containing wash buffer on the removal of impurities from an IgG4-containing cell culture during affinity chromatography is investigated. Specifically, four varying pH of wash solutions are compared.

Clarified cell culture supernatants containing IgG4 is harvested by centrifugation and purified using an AC column, in particular a Protein A column (Millipore, Eshmuno A, 1 CV: 3 mL), according to the conditions described in Table 12.

TABLE 12
Operating Conditions for Protein A Column

| | | Rt | (CV) |
|---|---|---|---|
| Step | Buffer | (min) | Vol. |
| Rinse | 50 mM Tris-HAc, 150 mM NaCl, | 5 | 2 |
| | pH 7.4 | | |
| Sanitization | 0.1M NaOH | | 3 |
| Equilibration | 50 mM Tris-HAc, 150 mM NaCl, | | 3 |
| | pH 7.4 | | |
| Loading | NA | | NA |
| Wash 1 | 50 mM Tris-HAc, 150 mM NaCl, | | 3 |
| | pH 7.4 | | |
| Wash 2 | Variable (see Table 13) | | 3 |
| Wash 3 | 50 mM NaAc-HAc, pH 5.5 | | 3 |
| Elution | 50 mM NaAc-HAc, pH 3.5 | | 50-50 |
| | | | mAu/mm |
| Strip | 1M Acetic Acid | | 3 |

The equilibrated column is loaded with clarified harvest and is first washed with wash 1 solution, followed by a second wash with wash 2 solutions described in Table 14 below, and then elated at low pH. The eluate is analyzed for its antibody concentration by UV280, for HMW/LMW by analytical size exclusion chromatography (SEC) and for HCP content by enzyme-linked immunosorbent assay. The various wash solutions compared for the second wash are set forth below in Table 13.

TABLE 13
Imidazole Containing Wash Solution with Varying pH Values

| Buffer | CV |
|---|---|
| 50 mM NaAc-HAc, pH 5.0 | 3 |
| 50 mM NaAc-HAc, 500 mM Imidazole-HCl, pH 5.0 | 3 |
| 50 mM PB, pH 7.0 | 3 |
| 50 mM PB, 500 mM Imidazole, pH 7.0 | 3 |
| 50 mM Tris-HCl, pH 9.0 | 3 |
| 50 mM Tris-HCl, 500 mM Imidazole, pH 9.0 | 3 |

The process performance for the Protein A purification of monoclonal antibody using the three different pH of Histidine-containing wash solutions shown in Table 14.

TABLE 14
Comparison of Acidic to Basic and Physiological pH in Imidazole-containing Wash Solution

| | Yield | HMW | Main peak | LMW | HCP | PLBL2 |
|---|---|---|---|---|---|---|
| Buffer | (%) | (%) | (%) | (%) | (ppm) | (ppm) |
| 50 mM NaAc—HAc, pH5.0 | 103 | 14.1 | 85.9 | 0.0 | 35766 | 5 |
| 50 mM NaAc—HAc, 500 mM Imidazole-HCl, pH5.0 | 106 | 14.4 | 85.5 | 0.0 | 15970 | <2 |
| 50 mM PB, pH7.0 | 109 | 13.9 | 86.0 | 0.0 | 29306 | 4 |
| 50 mM PB, 500 mM Imidazole, pH7.0 | 106 | 14.9 | 85.0 | 0.0 | 19891 | <2 |
| 50 mM Tris-HCl, pH9.0 | 112 | 14.5 | 85.4 | 0.0 | 17800 | 3 |
| 50 mM Tris-HCl, 500 mM Imidazole, pH9.0 | 109 | 14.5 | 85.4 | 0.1 | 15024 | <2 |

As shown in Table 14, varying the pH from acidic (pH 5.0) to basic (pH 9.0) has no effect on yield and aggregate level compared with the non-Imidazole containing wash solution. It's evident that the Imidazole-based solution is significantly efficient in removing HCPs at low pH, emphasizing the efficacy of the Imidazole-containing wash solution at acidic pH for affinity chromatography. Furthermore, in this case, it is clear that Imidazole alone does result in the desired HCP removal and it's only effective in composition with low pH.

### Example 6: Comparison of Histidine Wash Solution to Other Amino Acid Wash

In this example, the effect of Histidine-containing wash solution on the removal of impurities from an IgG4-containing cell culture during affinity chromatography is assessed. Specifically, seven wash solutions are compared.

Clarified cell culture supernatants containing IgG4 is harvested by centrifugation and purified using an AC column, in particular a Protein A column (Millipore, Eshmuno A, 1 CV: 3 mL), according to the conditions described below in Table 15.

TABLE 15
Operating Condition for Protein A column

| | | Rt | (CV) |
|---|---|---|---|
| Step | Buffer | (min) | Vol. |
| Rinse | 50 mM Tris-HAc, 150 mM NaCl, | 5 | 2 |
| | pH 7.4 | | |
| Sanitization | 0.1M NaOH | | 3 |
| Equilibration | 50 mM Tris-HAc, 150 mM NaCl, | | 3 |
| | pH 7.4 | | |
| Loading | NA | | NA |
| Wash 1 | 50 mM Tris-HAc, 150 mM NaCl, | | 3 |
| | pH 7.4 | | |
| Wash 2 | Variable (see Table 13) | | 3 |
| Wash 3 | 50 mM NaAc-HAc, pH 5.5 | | 3 |
| Elution | 50 mM NaAc-HAc, pH 3.5 | | 50-50 |
| | | | mAu/mm |
| Strip | 1M Acetic Acid | | 3 |

The equilibrated column is loaded with clarified harvest and is first washed with wash 1 solution, followed by a second wash with wash 2 solution described in Table 16, and then eluted at low pH. The eluate is analyzed for its antibody concentration by UV280, for HMW/LMW by analytical size exclusion chromatography (SEC) and for HCP content by enzyme-linked immunosorbent assay. The various wash solutions compared for the second wash are shown in Table 17.

TABLE 16
Wash Solution with Varying Components for Second Wash

| Buffer | CV |
|---|---|
| Without wash 2 | 3 |
| 50 mM NaAc-HAc, 1M NaCl, pH 5.5 | 3 |
| 50 mM NaAc-HAc, 0.5M Imidazole-HCl, pH 5.5 | 3 |
| 50 mM NaAc-HAc, 0.5M Histidine-HCl, pH 5.5 | 3 |
| 50 mM NaAc-HAc, 0.5M Arginine-HCl, pH 5.5 | 3 |
| 50 mM NaAc-HAc, 0.5M Proline, pH 5.5 | 3 |
| 50 mM NaAc-HAc, 0.5M L-Cysteine, pH 5.5 | 3 |
| 50 mM NaAc-HAc, 0.5M Serine, pH 5.5 | 3 |

The process performance for the Protein A purification of monoclonal antibody using the seven different wash solutions are shown below in Table 17.

TABLE 17
Comparison of Different Wash Solution for IgG4 Antibody

| | Yield | HMW | Main peak | LMW | HCP | PLBL2 |
|---|---|---|---|---|---|---|
| Buffer | (%) | (%) | (%) | (%) | (ppm) | (ppm) |
| Without wash 2 | 105 | 14.5 | 85.5 | 0.1 | 34409 | 8 |
| 50 mM NaAc—HAc, 1M NaCl, pH 5.5 | 105 | 14.6 | 85.5 | 0.0 | 27517 | 5 |
| 50 mM NaAc—HAc, 0.5M Imidazole-HCl, pH 5.5 | 103 | 14.3 | 85.7 | 0.0 | 17407 | <2 |
| 50 mM NaAc—HAc, 0.5M Histidine-HCl, pH 5.5 | 104 | 14.4 | 85.5 | 0.1 | 14522 | <2 |
| 50 mM NaAc—HAc, 0.5M Arginine-HCl, pH 5.5 | 102 | 14.1 | 85.7 | 0.0 | 13340 | <2 |
| 50 mM NaAc—HAc, 0.5M Proline, pH 5.5 | 109 | 14.1 | 85.7 | 0.1 | 24885 | 5 |
| 50 mM NaAc—HAc, 0.5M L-Cysteine, pH 5.5 | 108 | 14.2 | 85.7 | 0.1 | 16008 | 6 |
| 50 mM NaAc—HAc, 0.5M Serine, pH 5.5 | 109 | 14.3 | 85.6 | 0.0 | 19310 | 5 |

As shown in Table 17, the results demonstrate that Histidine and Arginine showed significant HCPs removal compared to control. In addition, Cysteine and Serine also showed HCP removal compared to control.

### Examples 72 Comparison of Different Wash Solution for Bi-Specific Antibody

The cDNA sequence to express a bi-specific anti-CD3×CD19 antibody as disclosed in WO 2019/057124A1 was cloned into two vectors, which contained Blasticidin and Zeocin resistance markers, respectively. Stable transfection was performed using liposome. After transfection, cells were plated in 96-well plates in selective media (CD CHO media containing 9 μg/mL Blasticidin and 400 μg/mL Zeocin) for minipool selection. After about 2 weeks of minipool selection, the high-producing minipools were expanded individually. The minipools were cloned by one round of FACS, the clones were screened by fed-batch cultures in spin tubes. The selected cell clones are cultured, and harvest is clarified from the bi-specific anti-CD3×CD19 antibody containing cell culture.

In this example, the effect of Histidine/Imidazole-containing wash buffer on the removal of impurities from the bi-specific antibody containing cell culture during affinity chromatography is assessed.

Clarified cell culture supernatants containing the bi-specific antibody is harvested by filtration and purified using an AC column, in particular a Protein A column (Millipore, Eshmuno A, 1 CV: 3 mL), according to the conditions described below in Table 18.

TABLE 18
Operating Conditions for Protein A Column

| | | Rt | (CV) |
|---|---|---|---|
| Step | Buffer | (min) | Vol. |
| Rinse | 50 mM Tris-HAc, 150 mM NaCl, | 5 | 2 |
| | pH 7.4 | | |
| Sanitization | 0.1M NaOH | | 3 |
| Equilibration | 50 mM Tris-HAc, 150 mM NaCl, | | 3 |
| | pH 7.4 | | |
| Loading | NA | | NA |
| Wash 1 | 50 mM Tris-HAc, 150 mM NaCl, | | 3 |
| | pH 7.4 | | |
| Wash 2 | Variable (see Table 19) | | 3 |
| Wash 3 | 50 mM NaAc-HAc, pH 5.5 | | 3 |
| Elution | 50 mM NaAc-HAc, pH 3.5 | | 50-50 |
| | | | mAu/mm |
| Strip | 1M Acetic Acid | | 3 |

The equilibrated column is loaded with clarified harvest and is first washed with wash 1 solution, followed by a second wash with wash 2 solutions described in Table 19, and then eluted at low pH. The eluate is analyzed for its antibody concentration by UV280, for HMW/LMW by analytical size exclusion chromatography (SEC) and for HCP content by enzyme-linked immunosorbent assay. The various wash solutions compared for the second wash are shown below in Table 19.

TABLE 19
Wash Solution with Varying Components for Second Wash

| Buffer | CV |
|---|---|
| Without wash 2 | 3 |
| 50 mM NaAc-HAc, 0.5M Histidine, pH 5.5 | 3 |
| 50 mM NaAc-HAc, 1M NaCl, pH 5.5 | 3 |
| 50 mM NaAc-HAc, 0.5M Arginine-HCl, pH 5.5 | 3 |

The process performance for the Protein A purification of bispecific antibody using different wash solutions are shown below in Table 20.

TABLE 20
Comparison of Wash Solutions Components on Purification Process Performance

| | Yield | HMW | Main peak | LMW | HCP | PLBL2 |
|---|---|---|---|---|---|---|
| Buffer | (%) | (%) | (%) | (%) | (ppm) | (ppm) |
| Without wash 2 | 90 | 7.5 | 90.0 | 2.5 | 5145 | 210 |
| 50 mN NaAc—HAc, 0.5M Histidine-HCl, pH5.5 | 89 | 7.8 | 89.9 | 2.5 | 2165 | 93 |
| 50 mM NaAc—HAc, 1M NaCl, pH5.5 | 91 | 8.0 | 89.6 | 2.4 | 2796 | 207 |
| 50 mM NaAc—HAc, 0.5M Arginine-HCl, pH5.5 | 86 | 10.0 | 87.1 | 2.9 | 1744 | NA |

As shown in Table 20, the results demonstrate that different wash 2 solutions have no effect on step yield and aggregate level while maintaining the higher recovery of product. Moreover, using a Histidine-containing wash solution at concentration of 500 mM results in 2.5-fold reduction of HCPs, a2-fold reduction of PLBL2, as compared to control.

### Examples 8: Comparison of Histidine/Imidazole Wash Solution to Other Wash Solutions

The cDNA sequence to express a monoclonal antibody targeting PD1 (Keytruda) disclosed in Patent application No.: WO2008/156712 A. The cDNA sequence to express a fusion protein targeting VEGF (Eylea) is disclosed in U.S. Pat. No. 7,070,959B1. The cDNA sequence was cloned into two vectors, which contained Blasticidin and Zeocin resistance markers, respectively. Stable transfection was performed using liposome. After transfection, cells were passaged in selective media (CD CHO media containing 9 μg/mL Blasticidin and 400 μg/mL Zeocin) for pool selection. After about 2 weeks of pool selection, the pools were cloned by FACS sorting. The clones were screened by fed-batch cultures in spin tubes. The selected cell clones are cultured, and harvest is clarified from anti-PD1 IgG4 or anti-VEGF fusion protein containing cell culture.

In this Example, the effect of Imidazole containing wash solution on the removal of impurities from clarified harvest from anti-PD1 IgG4 or anti-VEGF fusion protein containing cell culture during affinity chromatography is assessed. Specifically, four wash solutions are compared: one containing 1 M NaCl at pH 5.5, the second containing 0.5 M Histidine at pH 5.5, the third containing 0.5 M Arginine at pH 5.5, the fourth containing 0.5 M Imidazole at pH 5.5.

Clarified harvest from cell culture supernatants containing anti-PD1 IgG4 or anti-VEGF fusion protein is harvested by centrifugation and purified using an AC column, in particular a Protein A column (Millipore, Eshmuno A, 1 CV: 3 mL), according to the conditions described below in Table 21. The load capacity is 30 g/L for anti-PD1 IgG4 and 19 g/L for anti-VEGF fusion protein.

TABLE 21
Operating Conditions for Protein A Column

| | | Rt | (CV) |
|---|---|---|---|
| Step | Buffer | (min) | Vol. |
| Rinse | 50 mM Tris-HAc, 150 mM NaCl, | 5 | 2 |
| | pH 7.4 | | |
| Sanitization | 0.1M NaOH | | 3 |
| Equilibration | 50 mM Tris-HAc, 150 mM, NaCl, | | 3 |
| | pH 7.4 | | |
| Loading | NA | | NA |
| Wash 1 | 50 mM Tris-HAc, 150 mM NaCl, | | 3 |
| | pH 7.4 | | |
| Wash 2 | Variable | | 3 |
| Wash 3 | 50 mM NaAc-HAc, pH 5.5 | | 3 |
| Elution | 50 mM NaAc-HAc, pH 3.5 | | 50-50 |
| | | | mAu/mm |
| Strip | 1M Acetic Acid | | 3 |

The equilibrated column is loaded with clarified harvest and is first washed with wash 1 solution, followed by a second wash with wash 2 solution described in Table 22 or 23, and then eluted at low pH. The eluate is analyzed for its antibody or fusion protein concentration by UV 280, for HMW/LMW by analytical size exclusion chromatography (SEC) and for HCP content by enzyme-linked immunosorbent assay. The various wash solutions compared for the second wash are shown in Table 22 or 23.

TABLE 22
Wash Solution with Varying Components
for Second Wash (anti-PD1, IgG4)

| | Capacity | |
|---|---|---|
| Buffer | (g/L) | CV |
| Control (without wash 2) | 30 | 3 |
| 50 mM NaAc-HAc, 1M NaCl, pH 5.5 | | 3 |
| 50 mM NaAc-HAc, 0.5M Histidine-HCl, pH 5.5 | | 3 |
| 50 mM NaAc-HAc, 0.5M Arginine-HCl, pH 5.5 | | 3 |
| 50 mM NaAc-HAc, 0.5M Imidazole-HCl, pH 5.5 | | 3 |

TABLE 23
Wash Solutions with Varying Components for
Second Wash (anti-VEGF fusion protein)

| | Capacity | |
|---|---|---|
| Buffer | (g/L) | CV |
| Control (without wash 2) | 19 | 3 |
| 50 mM NaAc-HAc, 1M NaCl, pH 5.5 | | 3 |
| 50 mM NaAc-HAc, 0.5M Histidine-HCl, pH 5.5 | | 3 |
| 50 mM NaAc-HAc, 0.5M Arginine-HCl, pH 5.5 | | 3 |
| 50 mM NaAc-HAc, 0.5M Imidazole-HCl, pH 5.5 | | 3 |

The process performance for the Protein A purification of anti-PD1 IgG4 and anti-VEGF fusion protein using the four different wash solutions are shown in Table 24 and 25, separately.

TABLE 24
Comparison of Histidine Wash Solution
to Other Wash Solutions (anti-PD1 IgG4)

| | | | Main | | |
|---|---|---|---|---|---|
| | Yield | HMW | peak | LMW | HCP |
| Buffer | (%) | (%) | (%) | (%) | (ppm) |
| Control (without wash 2) | 97.5 | 1.5 | 98.6 | ND | 1878 |
| 50 mM NaAc-HAc, 1M NaCl, | 96.8 | 1.5 | 98.5 | ND | 1300 |
| pH 5.5 | | | | | |
| 50 mM NaAc-HAc, 0.5M | 95.9 | 1.5 | 98.5 | ND | 792 |
| Histidine-HCl, pH 5.5 | | | | | |
| 50 mM NaAc-HAc, 0.5M | 93.5 | 1.5 | 98.5 | ND | 768 |
| Arginine-HCl, pH 5.5 | | | | | |
| 50 mM NaAc-HAc, 0.5M | 94.3 | 1.4 | 98.6 | ND | 924 |
| Imidazole-HCl, pH 5.5 | | | | | |

TABLE 25
Comparison of Histidine Wash Solution to Other
Wash Solutions (anti-VEGF fusion protein)

| | | | Main | | |
|---|---|---|---|---|---|
| | Yield | HMW | peak | LMW | HCP |
| Buffer | (%) | (%) | (%) | (%) | (ppm) |
| Control (without wash 2) | 94.6 | 0.8 | 98.9 | 0.2 | 2238 |
| 50 mM NaAc-HAc, 1M NaCl, | 71.3 | 0.9 | 98.8 | 0.3 | 1488 |
| pH 5.5 | | | | | |
| 50 mM NaAc-HAc, 0.5M | 92.8 | 0.9 | 98.8 | 0.3 | 1145 |
| Histidine-HCl, pH 5.5 | | | | | |
| 50 mM NaAc-HAc, 0.5M | 96.3 | 0.7 | 98.8 | 0.6 | 1947 |
| Arginine-HCl, pH 5.5 | | | | | |
| 50 mM NaAc-HAc, 0.5M | 97.9 | 0.8 | 98.1 | 0.2 | 1652 |
| Imidazole-HCl, pH 5.5 | | | | | |

As shown in Table 24 and 25, the results demonstrate that different wash 2 solutions have no effect on step yield and aggregate level while maintaining the higher recovery of product. Also, it should be noticed that NaCl wash showed no significant decrease in HCPs compared with control. However, the Histidine and Imidazole wash solutions showed significant HCPs removal compared to the NaCl wash and control. Moreover, Histidine and Imidazole showed the similar ability in HCPs removal with Arginine solution.

### Example 9: Comparison of Different Wash Solution for HSA (Human Serum Albumin) Fusion Protein

The cDNA sequence to express anti-CD40 HSA fusion protein as disclosed in U.S. patent application Ser. No. 15/557,358 (see SEQ ID NO: 145) was cloned and is expressed in Chinese hamster ovary (CHO) cells using techniques familiar to those trained in the art.

In this example, the concentration and pH effect of Histidine or imidazole-containing wash buffer on the removal of impurities from HSA fusion protein-containing cell culture during affinity chromatography is investigated. Specifically, four varying concentration of wash solutions (0.1M, 0.3M, 0.5M, and 0.7M) with different pH are compared.

Clarified cell culture supernatants containing HSA fusion protein is harvested by centrifugation plus depth filtration, and purified using an AC column, in particular a Capto Blue (High Sub) column (1 CV: 3 mL), according to the conditions described in Table 26.

TABLE 26
Operating Conditions for Capto Blue Column

| | | Rt | (CV) |
|---|---|---|---|
| Step | Buffer | (min) | Vol. |
| Rinse | Purified Water | 5 | 3 |
| Sanitization | 0.5M NaOH | 5 | 3 |
| Pre-Equilibration | 1M NaCl | 5 | 3 |
| Equilibration | 20 mM Bis-Tris, 60 mM NaCl, | 5 | 3 |
| | pH 6.0 | | |
| Loading | NA | 5 | NA |
| Wash 1 | 20 mM Bis-Tris, 60 mM NaCl, | 5 | 2 |
| | pH 6.0 | | |
| Wash 2 | Variable | 5 | 3 |
| Wash 3 | 50 mM Bis-Tris, pH 7.0 | 5 | 2 |
| Elution | 50 mM Bis-Tris, 25 mM caprylate, | 150 | 250-50 |
| | 10 mM EDTA, pH 7.0 | cm/h | mAu/mm |
| Strip | 20 mM Bis-Tris, 0.5M caprylate, | 5 | 2 |
| | pH 7.5 | | |

The equilibrated column is loaded with clarified harvest and is first washed with wash 1 solution, followed by a second wash with wash 2 solutions described in Table 27 below, and followed by a third wash, then eluted elution buffer. The eluate is analyzed for its concentration by UV280, for HMW/LMW by analytical size exclusion chromatography (SEC) and for HCP content by enzyme-linked immunosorbent assay. The various wash solutions compared for the second wash are set forth in Table 27.

TABLE 27
Wash Solution with Varying Histidine/Imidazole Concentration/pH
for Wash 2 (HSA fusion protein)

| | Capacity | |
|---|---|---|
| Wash 2 condition | (g/L) | CV |
| Without wash 2 | 173 | 3 |
| 50 mM NaAc-HAc, 0.5M NaCl, pH 5.0 | | 3 |
| 50 mM NaAc-HAc, 0.1M Histidine-HCl, pH 5.0 | | 3 |
| 50 mM NaAc-HAc, 0.3M Histidine-HCl, pH 5.0 | | 3 |
| 50 mM NaAc-HAc, 0.5M Histidine-HCl, pH 4.5 | | 3 |
| 50 mM NaAc-HAc, 0.5M Histidine-HCl, pH 5.0 | | 3 |
| 50 mM NaAc-HAc, 0.5M Histidine-HCl, pH 5.5 | | 3 |
| 50 mM NaAc-HAc, 0.7M Histidine-HCl, pH 5.0 | | 3 |
| 50 mM NaAc-HAc, 0.1M Imidazole-HCl, pH 5.5 | | 3 |
| 50 mM NaAc-HAc, 0.3M Imidazole-HCl, pH 5.5 | | 3 |
| 50 mM NaAc-HAc, 0.5M Imidazole-HCl, pH 5.5 | | 3 |
| 50 mM NaAc-HAc, 0.7M Imidazole-HCl, pH 5.5 | | 3 |

The process performance for the Capto Blue purification of the HAS-fusion protein using the four different concentration of Histidine or Imidazole-containing wash solutions shown in Table 28.

TABLE 28
Comparison of Varying Histidine/Imidazole
Concentration/pH in Wash Solution

| | | | Main | | |
|---|---|---|---|---|---|
| | Yield | HMW | peak | LMW | HCP |
| Buffer | (%) | (%) | (%) | (%) | (ppm) |
| Without wash 2 | 91.2 | 2.6 | 97.2 | 0.4 | 32911 |
| 50 mM NaAc-HAc, 0.5M | 85.3 | 0.7 | 99.2 | 0.1 | 19446 |
| NaCl, pH 5.0 | | | | | |
| 50 mM NaAc-HAc, 0.1M | 87.2 | 0.7 | 99.1 | 0.3 | 33399 |
| Histidine-HCl, pH 5.0 | | | | | |
| 50 mM NaAc-HAc, 0.3M | 87.3 | 1.2 | 98.6 | 0.3 | 12229 |
| Histidine-HCl, pH 5.0 | | | | | |
| 50 mM NaAc-HAc, 0.5M | 83.5 | 0.9 | 99.1 | 0.1 | 3885 |
| Histidine-HCl, pH 4.5 | | | | | |
| 50 mM NaAc-HAc, 0.5M | 81.9 | 0.8 | 99 1 | 0.1 | 1472 |
| Histidine-HCl, pH 5.0 | | | | | |
| 50 mM NaAc-HAc, 0.5M | 68.2 | 0.6 | 99.3 | 0.0 | 1053 |
| Histidine-HCl, pH 5.5 | | | | | |
| 50 mM NaAc-HAc, 0.7M | 71.5 | 0.5 | 99.4 | 0.1 | 438 |
| Histidine-HCl, pH 5.0 | | | | | |
| 50 mM NaAc-HAc, 0.1M | 89.7 | 0.7 | 99.0 | 0.2 | 32316 |
| Imidazole-HCl, pH 5.0 | | | | | |
| 50 mM NaAc-HAc, 0.3M | 87.6 | 2.3 | 97.6 | 0.2 | 15148 |
| Imidazole-HCl, pH 5.0 | | | | | |
| 50 mM NaAc-HAc, 0.5M | 83.2 | 0.9 | 99.0 | 0.0 | 3683 |
| Imidazole-HCl, pH 5.0 | | | | | |
| 50 mM NaAc-HAc, 0.7M | 79.1 | 0.5 | 99.4 | ND | 1037 |
| Imidazole-HCl, pH 5.0 | | | | | |

As shown in Table 28, the results demonstrate that Histidine and Imidazole solution are significantly efficient in removing HCPs. Higher Histidine and Imidazole concentration of wash solution results in lower HCP in the eluate pool. Higher wash solution pH containing Histidine or Imidazole results in lower HCP in the eluate pool but lower step yield.

1. A method for improving impurities removal in the protein purification by affinity chromatography, comprising the following steps:
1) loading a protein sample onto an affinity chromatography column,
2) washing the column with a wash buffer solution comprising a compound of the formula I and a pH-adjusting agent, 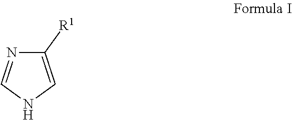
wherein, R¹ is H or C₁₋₆ alkyl; wherein C₁₋₆ alkyl is unsubstituted or substituted by one or two or three substituents independently selected from carboxy, amino, halogen or hydroxy.

2. The method of claim 1, wherein the compound is Histidine or Imidazole.

3. (canceled)

4. The method of claim 1, wherein the affinity chromatography is selected from Protein A chromatography, Capto Blue (High Sub) chromatography, Protein G chromatography, Protein L chromatography, Lambda Fab Select chromatography, Kappa Select chromatography, Ig Select chromatography, Blue Sepharose chromatography, Capto Heparin chromatography, VII Select chromatography, VIII Select chromatography, XSelect chromatography and Capto L chromatography.

5. The method of claim 1, wherein the pH-adjusting agent comprises at least one of acetate buffer such as NaAc and/or HAc, citrate buffer, Phosphate Buffer, Tris-HCl.

6. The method of claim 1, wherein the molarity of the compound is about 100 mM more.

7. The method claim 1, wherein pH of the wash buffer solution is about pH5.5 or less.

8. The method claim 1, wherein further not comprising elution step after the step 2).

9. The method claim 1, wherein the protein sample is an antibody comprising Fc domain or is a fusion protein.

10. The method of claim 17, wherein the Fc-fusion protein is composed of an Fc domain of IgG linked to a peptide or protein of interest.

11. The method of claim 17, wherein the HAS-fusion protein is composed of an HAS linked to a peptide or protein of interest.

12. The method of claim 1, wherein the impurities comprise host cell proteins (HCPs).

13. (canceled)

14. (canceled)

15. The method of claim 1, wherein the molarity of the compound is from about 100 mM to about 1 M.

16. The method of claim 1, wherein the molarity of the compound is from about 300 mM to 700 mM.

17. The method of claim 9, wherein the fusion protein is an Fc-fusion protein or a HAS-fusion protein.

18. A method for improving impurities removal in the protein purification by affinity chromatography, comprising the following steps:
1) loading a protein sample onto an affinity chromatography column,
2) washing the column with a wash buffer solution comprises Serine and/or Cysteine, and a pH-adjusting agent.

19. The method of claim 18, wherein the affinity chromatography is selected from Protein A chromatography, Capto Blue (High Sub) chromatography, Protein G chromatography, Protein L chromatography, Lambda Fab Select chromatography, Kappa Select chromatography, Ig Select chromatography, Blue Sepharose chromatography, Capto Heparin chromatography, VII Select chromatography, VIII Select chromatography, XSelect chromatography and Capto L chromatography.

20. The method of claim 18, wherein the pH-adjusting agent comprises at least one of acetate buffer such as NaAc and/or HAc, citrate buffer, Phosphate Buffer, Tris-HCl.

21. The method of claim 18, wherein the molarity of the compound is from about 300 mM to 700 mM.

22. The method of claim 18, wherein pH of the wash buffer solution is about pH5.5 or less.

23. The method of claim 1, wherein the protein sample is an antibody comprising Fc domain or is a fusion protein.
